(19) United States
(12) Patent Application Publication
(10) Pub. No.: US 2024/0177800 A1
(43) Pub. Date: May 30, 2024

(54) ALIGNMENT AND COMPARISON OF GENETIC INFORMATION

(71) Applicant: George Mason University, Fairfax, VA (US)

(72) Inventors: Lee Andrew Solomon, Fairfax, VA (US); Jacklin Yassa Sedhom, Centreville, VA (US); Jason Kinser, Bristow, VA (US)

(21) Appl. No.: 18/493,892

(22) Filed: Oct. 25, 2023

Related U.S. Application Data
(60) Provisional application No. 63/419,082, filed on Oct. 25, 2022.

(51) Int. Cl. G16B 15/30; G16B 30/10; G16B 35/20; G16B 40/10

(52) U.S. Cl. CPC G16B 15/30; G16B 30/10; G16B 35/20; G16B 40/10

(57) ABSTRACT

The present disclosure generally relates to methods and systems for identifying shared information between different DNA sequences, that bind the same protein, based on alignment of major groove hydrogen bonding between the different sequences. Such methods may be useful for designing novel DNA binding proteins, as well as identification of novel DNA protein binding consensus sequences, for use in gene therapies, treatment of diseases or disorders resulting from aberrant gene expression and/or cell proliferation, as well as pathogenic infections.

This application claims benefit and priority to U.S. Provisional Application No. 63/419,082, filed Oct. 25, 2022, which is incorporated herein by reference in its entirety.

### REFERENCE TO ELECTRONIC SEQUENCE LISTING

The application contains a Sequence Listing which has been submitted electronically in .XML format and is hereby incorporated by reference in its entirety. Said . XML copy, created on Feb. 14, 2024, is named “3013-12 US.xml” and is 33,904 bytes in size. The sequence listing contained in this .XML file is part of the specification and is hereby incorporated by reference herein in its entirety.

### TECHNICAL FIELD

The present disclosure generally relates to methods and systems for identifying shared information between different DNA sequences, that bind the same protein, based on alignment of major groove hydrogen bonding between the different sequences. Such methods may be useful for designing novel DNA binding proteins, as well as identification of novel DNA protein binding consensus sequences, for use in gene therapies, treatment of diseases or disorders resulting from aberrant gene expression and/or cell proliferation, as well as pathogenic infections.

### BACKGROUND

Protein-DNA binding is critically important for a number of biological processes (e.g. DNA transcription, replication, and repair) (1, 2). The sequence-specific interaction between proteins and DNA is of particular interest. Understanding the biophysical principles that guide how proteins recognize DNA with high specificity impacts how one studies regulatory processes in the living organisms and the ability to develop new gene therapies and therapeutic drugs (1). Many studies have investigated the complementarity of hydrogen bonds presented in the major groove (termed direct readout) (2-9). Luscombe et al. reviewed 129 protein-DNA complexes and clarified the roles of hydrogen bonds, van der Waals interactions, and water mediated bonds at the protein-DNA interface (3). Similarly, Garvie and Wolberger described how protein-DNA binding specificity arises from pairing hydrogen bond donors and acceptors between the protein and DNA and the role of van der Waals interaction between the thymine 5-position methyl group and amino acid side chains (3, 4). These studies were further corroborated by Emamjomeh et al., who showed that the highest degree of binding specificity is obtained from the complimentary pairing of hydrogen bond donors and acceptors in the major groove with amino acids (2). Recently, Lin and Guo carried out a comparative analysis for different protein-DNA complexes of different degrees of binding specificity (1). These studies all discussed the role of direct readout in recognition specificity, further highlighting the role of major groove hydrogen bonds.

However, these studies did not focus on proteins with multiple DNA-binding sites, what information is shared between them, or the minimal amount of direct readout needed. The previous studies were primarily focused on the base pairs themselves and did not seek to address how the information is displayed and if any of it is maintained between sequences. A drawback associated with focusing on specific nucleobases is that one can miss some of the individual hydrogen bonds essential for recognition and binding. Accordingly, methods are needed that can take a new view of direct readout, with a focus on proteins that bind multiple DNA sequences.

### SUMMARY

The present disclosure is generally directed to methods and systems, particularly computer-implemented processes for analyzing DNA-protein interactions. In certain embodiments. the methods are particularly useful for analyzing sequence data, e.g., nucleic acid sequence data for identifying individual hydrogen bonds essential for recognition and binding of proteins to DNA sequences. The disclosure provides methods, (e.g., computer-implemented methods) for identifying regions of a DNA sequence overlap between different DNA sequences that bind to the same DNA-binding protein. Specifically, the present disclosure generally relates to methods for determining the hydrogen bonds displayed by a target DNA sequence in the major groove that takes part in DNA-protein binding. The present disclosure is based on the development of an algorithm that converts a nucleotide sequence into an array of hydrogen bond donors and acceptors and methyl groups. The algorithm then aligns the non-covalent interaction arrays to identify what information is being maintained among multiple DNA sequences.

Specifically, a method is provided for identifying shared information between a DNA sequence and their DNA binding partner comprising one of more of the steps of: (i) obtaining publicly available the DNA sequences of corresponding DNA binding sites for the given DNA binding protein of interest for input into an algorithm to generate hydrogen bond patterns, the algorithm configured to map base pairs of the DNA sequences to preconfigured patterns, each of the preconfigured patterns comprising at least three of: an indication of a hydrogen bond donor (“HBD”), an indication of a hydrogen bond acceptor (“HBA”), an indication of a thymine methyl group (“TMG”), or an indication of none; (ii) converting the individual base pairs into a four-slot vertical array of designated hydrogen bond donors, acceptors, methyl groups or if nothing is in that position; (iii) aligning the hydrogen bond patterns to obtain one consensus pattern that is shared among all the protein binding sites; (iv) obtaining the crystal or NMR structures for the various protein-DNA complexes from the publicly available Protein Data Bank and verifying through the crystal and NMR structures that the maintained bonds in the alignment are indeed used by the protein for binding and recognition; and (v) obtaining the final refined patterns by aligning the verified contacts that were detected in the published structures for each binding site complex.

### BRIEF DESCRIPTION OF THE FIGURES

FIG. 1A-B. Hydrogen-bond donor/acceptor pattern exposed in the major groove. FIG. 1A. AT and GC base pairs showing which atoms contribute to the hydrogen bonding pattern. FIG. 1B. DNA sequences (SEQ ID NO. 26) displayed as an array of hydrogen bond acceptors (red circle) and donors (blue circle). The white circles represent the thymine methyl groups.

FIG. 2 (SEQ ID NOS. 1-3) Workflow diagram depicting the creation, alignment, verification and final realignment of the Lac repressor protein. Nucleotide numbering is taken from alignment output. Step 1 and 2: the disclosed algorithm takes input sequences and converts them into arrays of hydrogen bonds (blue circle: hydrogen bond donor, red circle: hydrogen bond acceptor, white circle: methyl group). Step 3: the algorithm aligns the various arrays to extract all possible information that is shared between the sequences. Step 4: PDB structures are used to verify which contacts are present in the actual protein-DNA interactions. All numbering of amino acids and nucleotides is taken from the PDB file's internal sequencing information. Step 5: the verified arrays are realigned to produce a final distinct pattern of information from all input sequences that is used by the protein itself.

FIG. 3A-B Lac operators sequences and their consensus pattern. FIG. 3A. (SEQ ID NOS. 1-3) The sequences of the three operators. The conserved bases are highlighted in grey, while the different bases which maintained some patterns are yellow highlighted. FIG. 3B. The consensus pattern that resulted from aligning the three sequences' hydrogen bond donors and acceptors pattern. The red circle represents hydrogen bond acceptor atom, the blue circle represents hydrogen bond donor atom, and the white circle represents the methyl group.

FIG. 4A-B. The verified consensus pattern of hydrogen bonds and interactions among the three operator sites for the lac repressor. FIG. 4A. (SEQ ID NOS. 1-3) The three operators' sequences are color coded. Black letters indicate base pairs that are different among the three operators and do not have any maintained hydrogen bonds. Green letters indicate the conserved base pairs which have direct bonds and contacts with the lac repressor shared among the three operators. Blue letters are conserved base pairs which don't have any bonds or contacts shared among the three operators. Red letters indicate different base pairs in the three operators which contribute with the same bonds to the binding with lac repressors. FIG. 4B. The distinct pattern of bonds and contacts shared among the three operators' complexes. The representation of colored circles is the same as in FIG. 3B.

FIG. 5A-C. The alignment of 2 Lac R operators O1 and O2. FIG. 5A. (SEQ ID NOS. 1-2) The 2 operators' sequences are color coded. Color coding is the same as in FIG. 4A-B. FIG. 5B. The distinct pattern of bonds and contacts shared between the 2 operators' complexes. The light blue circles represent the hydrogen bonds which are detected in the minor groove. The representation of colored circles is the same as in FIG. 3B. FIG. 5C. The hydrogen bond at location 14 verified from the NMR structures of the 2 operators.

FIG. 6A-C. The alignment of 2 Lac R operators O1 and O2. FIG. 6A. (SEQ ID NOS. 1-2) The 2 operators' sequences are color coded. Color coding is the same as in FIG. 4. FIG. 6B. The distinct pattern of bonds and contacts shared between the 2 operators' complexes. The representation of colored circles is the same as in FIG. 3B. FIG. 6C. the hydrogen bonds at location 16 verified from the NMR structures of the 2 operators.

FIG. 7A-C. The alignment of 2 Lac R operators O2 and O3. FIG.7A. (SEQ ID NOS. 2-3) The 2 operators' sequences are color coded. Color coding is the same as in FIG. 4. FIG.7B. The distinct pattern of bonds and contacts shared between the 2 operators' complexes. The representation of colored circles is the same as in FIG. 3B. FIG.7C. the hydrogen bonds at locations 13, 14 and 16 verified from the NMR structures of the 2 operators.

FIG. 8A-B. FIG. 8A. The consensus pattern of the aligned three operators that C-protein can bind. FIG. 8B. OLM consensus pattern of the aligned two operators that C-protein could recognize, O_{L} and O_{M}. The representation of colored circles is the same as in FIG. 3B.

FIG. 9A-C. The distinct pattern of bonds and interactions that is common between O_{L} and O_{M} in the OLM consensus pattern. FIG. 9A. (SEQ ID NOS. 5 and 7) Color coding is the same as in FIG. 4. FIG. 9B. The distinct pattern of bonds and contacts shared between the two operators. The representation of colored circles is the same as in FIG. 3B. FIG. 9C. the hydrogen bonds at locations eight verified from the crystal structures of the two operators.

FIG. 10A-B. The distinct pattern of bonds and interactions that is highlighted from the crystal structures of the 3 operators complexes in the consensus pattern. FIG. 10A. (SEQ ID NOS. 5,7 and 6) The three operators' sequences are color coded. Color coding is the same as in FIG. 4A-B. FIG. 10B. The pattern of the bonds and the interactions highlighted from the crystal structures of their corresponding operators in the consensus pattern. The representation of colored circles is the same as in FIG. 3B.

FIG. 11A-B. CI repressor operators' sequences and their consensus pattern. FIG. 11A. (SEQ ID NOS. 8-13) The sequences of the six operators. The conserved bases are highlighted in grey while the variable bases which have common pattern are yellow highlighted. FIG. 11B. The consensus pattern results from aligning the six sequences' hydrogen bond donors and acceptors pattern. The representation of colored circles is the same as in FIG. 3B.

FIG. 12A-B. The distinct pattern of bonds and interactions is highlighted from the crystal structures of the six operators' complexes in the consensus pattern. FIG. 12A. (SEQ ID NOS. 8-13) The six operators' sequences are color coded. Color coding is the same as in FIG. 4A-B. FIG. 12B. The pattern of the bonds and the interactions highlighted from the crystal structures of their corresponding operators in the consensus pattern. The representation of colored circles is the same as in FIG. 3B.

FIG. 13A-C. The distinct pattern of bonds and interactions that is common among the three binding sites of the right side O_{R}. FIG. 13A. (SEQ ID NOS. 8-10)The three operators' sequences are color coded. Color coding is the same as in FIG. 4. FIG. 13B. The distinct pattern of bonds and contacts shared among the three operators. The representation of colored circles is the same as in FIG. 3B. FIG. 13C. the hydrogen bonds at location 7 verified from the crystal structures of the two operators O_{R}1 and O_{R}2.

FIG. 14A-C. The distinct pattern of bonds and interactions that is common among the three binding sites of the left side O_{L}. FIG. 14A. (SEQ ID NOS. 8-10) the three operators' sequences are color coded. Color coding is the same as in FIG. 4. FIG. 14B. The distinct pattern of bonds and contacts shared among the three operators. The representation of colored circles is the same as in FIG. 3B. FIG. 14C. The hydrogen bonds at locations 10, 11 and 12 verified from the crystal structures of the two operators O_{L}1 and O_{L}3.

FIG. 15A-C. A comparative analysis among the binding sites of the lambdoid phages: λ-phage, VT2-SA and Stx2 I. FIG. 15A. (SEQ ID NOS. 8, 14 and 15) The alignment of O_{R}1 sequences from the three strains. FIG. 15B. (SEQ ID NOS. 9, 16 and 17) the alignment of O_{R}2 sequences from the three strains. FIG. 15C. (SEQ ID NOS. 10, 18 and 19) The alignment of O_{R}3 from the three strains. See Fig S4 for O_{L} sites' alignments. The representation of colored circles is the same as in FIG. 3B.

FIG. 16A-D. FIG. 16A. The NMR structure of lac repressor O1 which has 20 conformers (PDB ID: 1LIM). FIG. 16B. The NMR structure of lac repressor O1 which has 20 conformers (PDB ID: 2KEI) FIG. 16C. the NMR structure of lac repressor O2 which has 20 conformers (PDB ID: 2KEJ). FIG. 16D. the NMR structure of lac repressor O3 which has 10 conformers (PDB ID: 2KEK).

FIG. 17A-B. The refined/collective consensus pattern of lac repressor-O2 complex. FIG. 17A. the 20 refined consensus pattern after verifying each single bond/contact from each individual conformer in 2KEI crystal structure. FIG. 17B. the collective refined pattern after summing up the patterns shown up in the 20 conformers. The light blue circles refer to hydrogen bonds donor atoms from the minor groove of O2 operator.

FIG. 18A-C. FIG. 18A. The verified bonds and interactions between Lac R and its operator O1 from the NMR structure of their complex. FIG. 18B. The verified bonds and interactions between Lac R and its operator O2 from the NMR structure of their complex. FIG. 18C. The verified bonds and interactions between Lac R and its operator O3 from the NMR structure of their complex. The light blue circles represent the hydrogen bonds made between the protein and the base pairs in the minor groove.

FIG. 19A-B. FIG. 19A. the bonds and interactions between OL and C-protein which are highlighted from their corresponding crystal structures of their complex in the OLM consensus pattern. FIG. 19B. the bonds and interactions between OM and C-protein which are highlighted from their corresponding crystal structure of their complex in the OLM consensus pattern.

FIG. 20A-C. A comparative analysis among the binding sites of the lambdoid phages: 2-phage, VT2-SA and Stx2 I. FIG. 20A. (SEQ ID NOS. 11, 20 and 21) the alignment of OL1 sequences from the three strains. FIG. 20B. (SEQ ID NOS. 12, 22 and 23) the alignment of OL2 sequences from the three strains. FIG. 20C. (SEQ ID NOS. 13, 24 and 25) the alignment of OL3 from the three strains.

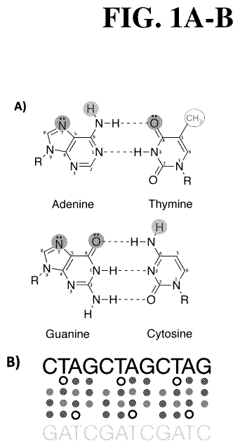

### DETAILED DESCRIPTION

Unless otherwise defined, all technical and scientific terms used herein have the same meaning as commonly understood by one skilled in the art. Although methods and materials similar to or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

DNA-binding proteins are defined herein are proteins that have DNA-binding domains that bind to specific DNA sequences. These sequence-specific proteins contain functional groups that identify base pairs and allow them to interact with B-DNA's major groove. Such DNA-binding proteins, include transcription factors which are involved in transcriptional regulation, such as transcription activators or repressors; DNA replications factors which are involved in the replication of whole genome or DNA fragments; repair factors that have a role in removing single base pairs or specific oligonucleotides and filling the gaps with suitable nucleotides; and histones which are involved in transcription and chromosome packaging in the cell nucleus. DNA-binding motifs involved in forming a DNA complex include, for example, Zinc finger regions, helix-turn-helix regions, and Leucine zippers.

The present disclosure generally relates to methods for determining the hydrogen bonds displayed by a target DNA sequence in the major-groove that take part in DNA-protein binding. The present disclosure is based on the development of an algorithm that converts a nucleotide sequence into an array of hydrogen bond donors and acceptors and methyl groups. The algorithm then aligns the non-covalent interaction arrays to identify what information is being maintained among multiple DNA sequences.

The general method which can be applied for a specific DNA-binding protein comprises the following steps. First, publicly available DNA sequences of corresponding DNA-binding sites for a given DNA-binding protein are obtained. Sources of such information include published literature as well as protein/DNA databases. The collected DNA-binding sites are then used as input into the novel algorithm disclosed herein to generate their corresponding hydrogen bond patterns. More specifically, the algorithm assigns a pattern containing hydrogen bond acceptors (e.g., designated “HBA”), hydrogen bond donors (e.g., designated “HBD”), thymine methyl group (e.g., designated “TMG”), and/or “None,” to each base pair. The algorithm assigns the patterns shown in the following Table 1.

| Base pair | Assigned Pattern |
|---|---|
| Adenine - Thymine | HBA, HBD, HBA, TMG |
| Thymine - Adenine | TMG, HBA, HBD, HBA |
| Cytosine - Guanine | None, HBD, HBA, HBA |
| Guanine - Cytosine | HBA, HBA, HBD, None |

As shown in the table, the pattern for an Adenine-Thymine base pair and the pattern for a Thymine-Adenine base pair are reverse of each other, and the pattern for a Cytosine-Guanine base pair and the pattern for a Guanine-Cytosine base pair are reverse of each other.

In a second step, the individual base pairs are converted into a four-slot vertical array of (i) designated hydrogen bond donors, (ii) acceptors, (iii) methyl groups or (iv) if nothing is in that position (i.e., the five-position of cytosine). In a specific embodiment disclosed herein, for illustration purposes, the individual base pairs are converted into a four-slot vertical array of hydrogen bond donors (blue circle), acceptors (red circle), methyl groups (white circle), or left blank if nothing is in that position (i.e., the five-position of cytosine). While methyl groups are only present on thymine nucleotides, they are included for further development of the algorithm which will include methylated nucleotides.

In a third step, the hydrogen bond patterns are aligned to obtain only one pattern that is shared among all the protein binding sites which is referred to herein as the “consensus pattern.” As disclosed herein, a 100% cutoff was held, meaning that a specific hydrogen bond had to be present in every sequence or it was not used. However, in another embodiment lower percent cut offs may be held.

In a fourth step, the crystal or NMR structures for the various protein-DNA complexes are obtained from the publicly available Protein Data Bank (PDB, www.rcsb.org). The crystal and NMR structures are then used to verify that the maintained bonds in the alignment are indeed used by the protein for binding and recognition. Any bonds and contacts not detected in the available structures are eliminated from the consensus pattern.

All the selected structures from PDB should satisfy one or more of the following conditions: high resolution crystal structures (up to 3.0 Å) which provides detailed information about protein-DNA interaction or NMR structures, the DNA strands have the sequence of the known binding sites, and non-mutated structures except for Lac R NMR structures which were mutated to link the dimeric Lac R headpiece covalently to facilitate the NMR studies. Structures with consensus sequences, palindromic DNA sequences, or any mutated DNA sequences were excluded from the analysis since the algorithm is built on analysis of the real and exact binding sites' sequences. Also, structures that have inducers or factors that affect the natural binding were excluded since the study is mainly concerned with analysis of the absolute conditions of binding that happens in nature without the presence of any external influences.

The “H-bonds” structural analysis tool, built into UCSF ChimeraX, was used to identify, and analyze the hydrogen bonds that formed between the protein and the DNA. Persons skilled in the art will understand the UCSF ChimeraX program and the “H-bonds” structural analysis tool. The numbering of amino acids and nucleotides herein are taken from the sequence information in the PDB file. The relax distance tolerance was 0.4 Å and the relax angle tolerance was 20.0° (20). However, such tolerances may range from [provide range]. In a specific embodiment, all the hydrogen bonds that were detected using the previous criteria were kept, even if two hydrogen bonds were detected from the same atom, to avoid any user-bias of the results. The “Swapna” command in UCSF Chimera mutates one nucleic acids base to another. Persons skilled in the art will understand the UCSF Chimera program and the “Swapna” command. After making the required mutations for the DNA strands in the protein-complex, the energy minimization function in UCSF Chimera was used to relax the entire complex structure. UCSF Chimera uses the AMBER forcefield to minimize protein structures. First, it performs Steepest descent minimization to relieve highly unfavorable clashes. Then, it performs conjugate gradient minimization to reach an energy minimum. The parameters for energy minimization were steepest descent steps: 100, steepest descent step size: 0.02 Å, conjugate gradient steps: 10, conjugate gradient step size: 0.02 Å, update interval: 10 and no atoms were fixed.

The “Contacts” structural analysis tool was used to detect van der Waals interactions between the methyl group of thymine and hydrophobic groups on amino acids. Persons skilled in the art will understand the “Contacts” structural analysis tool of the UCSF ChimeraX program. The focus was on amino acid residues that are directly contacting the DNA. Any interchain interactions, that were not included in DNA binding, were ignored, however, bonds were often identified by the software. Again, to avoid bias those were left in but were not considered in further analyses.

In a fifth step the final refined patterns were obtained by aligning the verified contacts that were detected in the published structures for each binding site complex. This provided the final “distinct pattern” of the common hydrogen bonds and van der Waals contacts that formed between a cognate protein and its different binding sites.

Many physiological and pathophysiological processes can be controlled by the selective up or down regulation of gene expression. Examples of pathologies that may be controlled by selective transcriptional regulation include cancer, autoimmunity, neurological disorders, developmental syndromes, diabetes, cardiovascular disease and obesity. among others. In addition, pathogenic organisms such as viruses, bacteria, fungi, and protozoa could be controlled by altering gene expression.

Thus, there is a clear need for therapeutic approaches that are able to up-regulate beneficial genes and down-regulate disease causing genes. DNA-binding domains may be engineered to increase the scope, specificity, and usefulness of these binding proteins for a variety of applications including engineered transcription factors for regulation of endogenous genes in a variety of cell types and engineered nucleases that can be similarly used in numerous models, diagnostic and therapeutic systems, and all manner of genome engineering and editing applications.

Accordingly, the methods disclosed herein may be utilized for development of novel DNA-binding proteins, e.g., artificial transcription factors or replication factors. The methods may be used to better screen out the minimal required information and target those hydrogen bond partners when looking at the interface. This could lead to transcription factors with specificity toward multiple sequences as well as a deeper understanding of how existing ones recognize their target DNA. Artificial nucleases, which link the cleavage domain of a nuclease to a designed DNA-binding protein (e.g., zinc-finger protein (ZFP) linked to a nuclease cleavage domain such as from FokI), may be used for targeted cleavage in cells.

Accordingly, the present disclosure provides methods for identifying an engineered DNA-binding protein that binds to a target DNA sequence of interest, said method comprises one or more of the steps of : (i) obtaining publicly available target DNA sequences of interest corresponding to DNA binding sites for the engineered DNA binding protein for input into an algorithm to generate hydrogen bond patterns, the algorithm configured to map base pairs of the DNA sequences to preconfigured patterns, each of the preconfigured patterns comprising at least three of: an indication of a hydrogen bond donor (“HBD”), an indication of a hydrogen bond acceptor (“HBA”), an indication of a thymine methyl group (“TMG”), or an indication of none; (ii) converting the individual base pairs into a four-slot vertical array of designated hydrogen bond donors, acceptors, methyl groups or if nothing is in that position; (iii) aligning the hydrogen bond patterns to obtain one consensus pattern that is shared among all the protein binding sites; (iv) obtaining the crystal or NMR structures for the protein-DNA complexes and verifying through the crystal and NMR structures that the maintained bonds in the alignment are indeed used by the protein for binding and recognition; and (v) obtaining the final refined patterns by aligning the verified contacts that were detected in the published structures for each binding site complex, thereby identifying an engineered DNA-binding protein that binds to a target DNA sequence of interest.

Said target DNA sequence of interest may, for example, be a sequence that is known to regulate the expression of a gene of interest. In an embodiment, the target DNA binding sites may be, for example, know promoter sequences or DNA-binding motifs involved in forming a DNA complex include, for example, Zinc finger regions, helix-turn-helix regions, and Leucine zippers. The DNA-binding proteins may be an activator or repressor of transcription. The engineered DNA-binding protein may be a genetically engineered fusion protein that targets a specific activity, e.g., enzyme activity, to a DNA-binding site of interest.

The present disclosure also provides a method for identifying suitable DNA target sequences for an engineered DNA-binding protein of interest. Accordingly, a method is provided comprising the steps of: (i) collecting possible suitable DNA target sequences corresponding to DNA-binding sites for a given DNA-binding protein of interest for input into an algorithm to generate hydrogen bond patterns, the algorithm configured to map base pairs of the DNA sequences to preconfigured patterns, each of the preconfigured patterns comprising at least three of: an indication of a hydrogen bond donor (“HBD”), an indication of a hydrogen bond acceptor (“HBA”), an indication of a thymine methyl group (“TMG”), or an indication of none; (ii) converting the individual base pairs into a four-slot vertical array of designated hydrogen bond donors, acceptors, methyl groups or if nothing is in that position; (iii) aligning the hydrogen bond patterns to obtain one consensus pattern that is shared among all the protein binding sites; (iv) obtaining the crystal or NMR structures for the protein-DNA complexes and verifying through the crystal and NMR structures that the maintained bonds in the alignment are indeed used by the protein for binding and recognition; and (v) obtaining the final refined patterns by aligning the verified contacts that were detected in the published structures for each binding site complex, thereby identifying suitable DNA target sequences for DNA-binding proteins.

The present disclosure also provides methods of targeted manipulation of gene expression utilizing the engineered DNA-binding proteins identified using the methods disclosed herein. In some embodiments, the engineered DNA-binding proteins include engineered transcription factors and or DNA-binding proteins having enzymatic activity, e.g., DNA cleavage activity. For example, a method is provided for modulating the expression of an endogenous cellular gene in a cell, the method comprising the steps of contacting a first target site in the endogenous cellular gene with an engineered DNA-binding protein thereby modulating expression of the endogenous cellular gene.

The engineered DNA-binding proteins, identified using the methods disclosed herein, as well as nucleic acids encoding the DNA-binding proteins, are also provided as are pharmaceutical compositions. In addition, included are host cells, cell lines and transgenic organisms (e.g., plants, fungi, animals) comprising the DNA-binding proteins and/or encoding nucleic acids.

In a specific embodiment, based on the observed binding of the test DNA-binding protein of interest to the identified “distinct pattern”, the DNA sequence and/or the DNA-binding protein may be genetically engineered to, for example, increase the binding specificity and/or affinity between the DNA-binding protein and the DNA sequence.

### EXAMPLE

### Programs Used for Visualization and Alignment

For visualization of the crystal and NMR structures, as well as inspection of bonds and interactions, both UCSF Chimera (15) and UCSF ChimeraX (16, 17) were chosen for these studies because they can be learned quickly, and are available free of charge for noncommercial use. The analysis algorithm was developed using Python with packages NumPy (18) and Matplotlib (19). The codes are available on the GitHub page. Implementing the algorithm using Python is merely an example, and persons skilled in the art will understand how to implement the algorithm using other programming languages and/or in other ways.

### The General Method Applied for Each DNA-Binding Protein

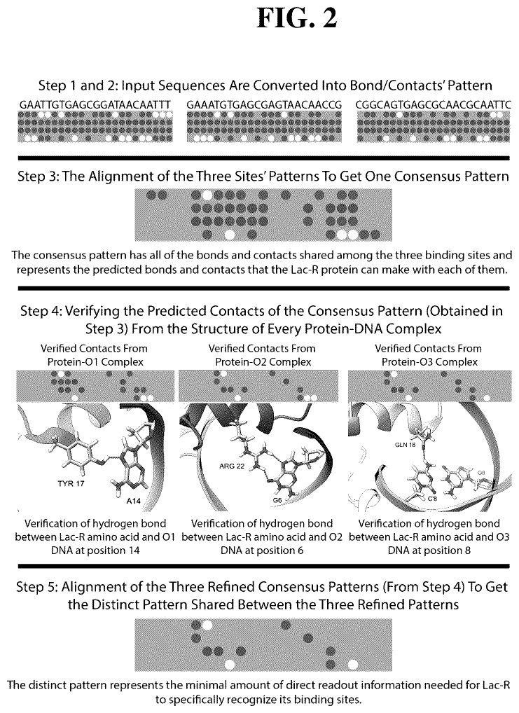

The general workflow can be found in FIG. 2. In Step 1 and 2 the sequences of all the corresponding DNA-binding sites were obtained from the literature and used as input into the algorithm to generate their corresponding hydrogen bond patterns. Individual base pairs were converted into a four-slot vertical array of hydrogen bond donors (blue circle), acceptors (red circle), methyl groups (white circle), or left blank if nothing was in that position (i.e. the five-position of cytosine). While methyl groups are only present on thymine nucleotides, they are included for further development of this algorithm which will include methylated nucleotides.

In Step 3, these hydrogen bond patterns were aligned to obtain only one pattern that is shared among all the binding sites (consensus pattern). A 100% cutoff was held, meaning that a specific hydrogen bond had to be present in every sequence or it was not used.

Step 4: The crystal or NMR structures for the various protein-DNA complexes were obtained from the Protein Data Bank (PDB, www.rcsb.org). These structures were used to verify that the maintained bonds in the alignment are indeed used by the protein for binding and recognition. Any bonds and contacts not detected in the available structures were eliminated from the consensus pattern. The “H-bonds” structural analysis tool, built into UCSF ChimeraX, was used to identify and analyze the hydrogen bonds that formed between the protein and the DNA. The numbering of amino acids and nucleotides disclosed herein is taken from the sequence information in the PDB file. The relax distance tolerance was 0.4 Å and the relax angle tolerance was 20.0° (20). All the hydrogen bonds that were detected using the previous criteria were kept, even if two hydrogen bonds were detected from the same atom, to avoid any user-bias of the results. The “Contacts” structural analysis tool was used to detect van der Waals interactions between the methyl group of thymine and hydrophobic groups on amino acids. The focus was on amino acid residues that are directly contacting the DNA. Any interchain interactions, that were not included in DNA-binding, were ignored, however, bonds were often identified by the software. Again, to avoid bias in the results those were left in but were not considered in further analyses.

Step 5: The final refined patterns were obtained by aligning the verified contacts that were detected in the published structures for each binding site complex. This provided the final “distinct pattern” of the common hydrogen bonds and van der Waals contacts that formed between a cognate protein and its different binding sites.

### Criteria for PDB Structure Selection

All the selected structures from PDB should satisfy the following conditions: high resolution crystal structures (up to 3.0 Å) which provides detailed information about protein-DNA interaction or NMR structures, the DNA strands have the sequence of the known binding sites, and non-mutated structures except for Lac R NMR structures which were mutated to link the dimeric Lac R headpiece covalently to facilitate the NMR studies (21). Structures with consensus sequences, palindromic DNA sequences, or any mutated DNA sequences were excluded from the analysis since the algorithm is built on analysis of the real and exact binding sites' sequences. Also, structures that have inducers or factors that affect the natural binding were excluded since the study is mainly concerned with analysis of the absolute conditions of binding that happens in nature without the presence of any external influences.

### Nucleic Acid Mutations and Energy Minimization

The “Swapna” command in UCSF Chimera mutates one nucleic acids base to another. After making the required mutations for the DNA strands in the protein-complex, the energy minimization function in UCSF Chimera was used to relax the entire complex structure. UCSF Chimera uses the AMBER forcefield to minimize protein structures. First, it performs Steepest descent minimization to relieve highly unfavorable clashes. Then, it performs conjugate gradient minimization to reach an energy minimum. The parameters for energy minimization were steepest descent steps: 100, steepest descent step size: 0.02 Å, conjugate gradient steps: 10, conjugate gradient step size: 0.02 Å, update interval: 10 and no atoms were fixed.

### Results

### Lac R-DNA Specific Binding Results and Data Analysis

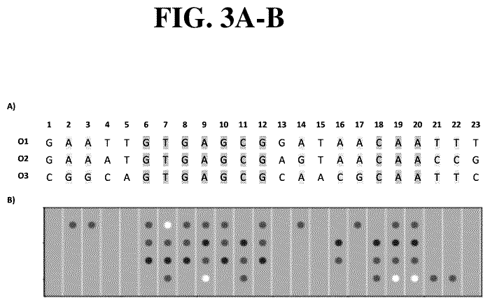

The Lac R protein controls the transcription of lactose metabolizing genes (21-24). Transcription is repressed by Lac R binding, as a dimer, to its operator site O1(21, 25). Repression is further enhanced by binding to the two auxiliary operator sites O2 or O3 (21). The binding affinity of Lac R is highest for O1 followed by O2, and finally O3 (21). The three sequences of the operator sites were obtained from the literature (Table 1) (21). The contacts arrays derived from these sequences were aligned to produce an initial pattern (FIG. 3), which shows that the bases in positions 6-12 and 18-20 are entirely conserved throughout the three operator sequences (highlighted in grey). Some positions had no common information and appeared as empty columns (locations 1, 4, 5, 13, 15 and 23, FIG. 3). However, it was noted that in locations 2, 3, 14, 16, 17, 21 and 22, the hydrogen bonds are maintained, but the base pair identity is different. These are highlighted in yellow as in (FIG. 3).

The available structures for Lac R operator complexes were obtained from the PDB. Four NMR structures were used: two structures for Lac R-O1 complex (PDB ID: 2KEI and 1L1M) (21, 25), one structure for Lac R-O2 complex (PDB ID: 2KEJ) (21) and one structure for Lac R-O3 complex (PDB ID: 2KEK)(21). The predicted bonds and contacts were verified from the consensus sequence using the NMR structures.

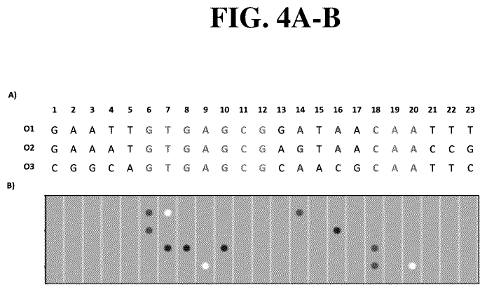

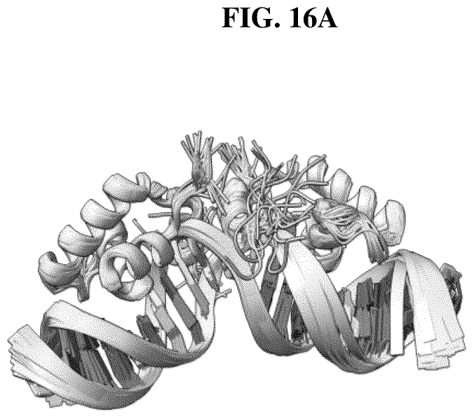

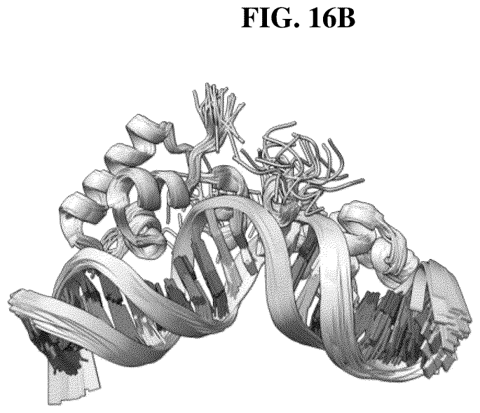

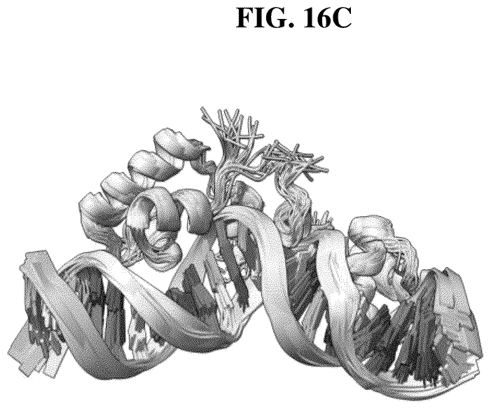

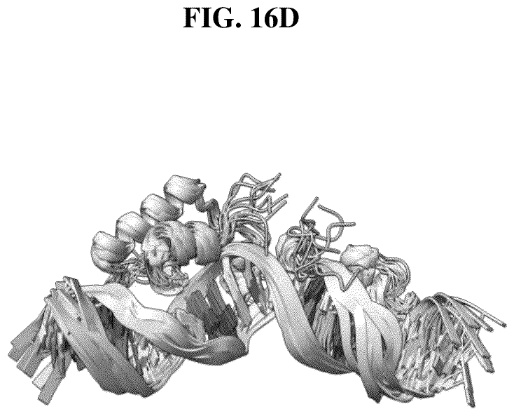

The structure of lac R-O1 complex showed 20 bonds and interactions in the major groove in the consensus pattern (FIG. 16A), while the NMR structures of O2 and O3 protein-DNA complexes show 13 hydrogen bonds and methyl group interactions (FIG. FIG. 16B, C). By comparing all the refined hydrogen bonds and interactions that lac repressor can make with each of the three operator sequences, a distinct pattern was extracted (FIG. 4B). It is believed that the distinct pattern represents the minimal number of specific bonds and contacts Lac R needed to recognize these three binding sites.

This distinct pattern was then analyzed to see what base pairs could make these bonds and interactions to Lac R protein. It was found that most of the interactions came from conserved base pairs among the 3 operators (FIG. 4A, green colored). However, in locations 14 and 16 (red colored), Lac R made hydrogen bonds despite different base pairs being present at these positions. In addition, three base pairs were maintained in the three operator sites in positions 11, 12 and 19, but no observe common hydrogen bonding to Lac R protein maintained in the three operators (blue colored) was seen. These results indicate that Lac R recognizes specific hydrogen bonds in the same location of all three operators regardless of the base pair identity.

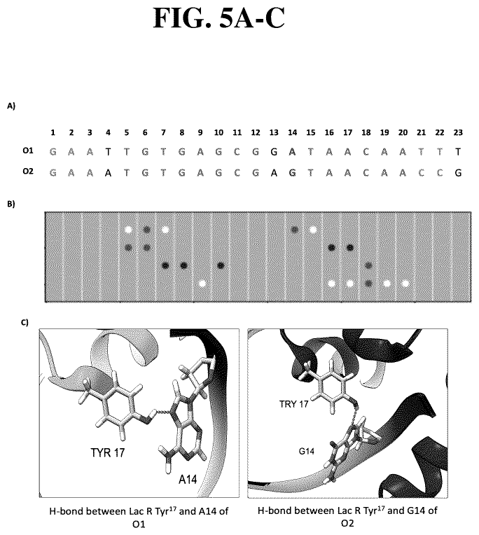

A deeper analysis aligning two binding sites together was run to see how the information changes between individual operators and if that can shed any light on the order of binding. O1 and O2, O1 and O3, and finally O2 and O3 were compared. The indispensable operator O1 was chosen over the auxiliary operator O2. These 2 sequences are the same except for four nucleobases at locations 4, 13, 14 and 23 (FIG. 5A). The verified pattern of hydrogen bonds for operators O1 and O2 was extracted (FIG. 5B). The distinct pattern indicates that all the bonds and contacts are made from the conserved nucleobases in the two binding sites except for one bond at location 14 that was maintained despite the change in the base pair identity from A in binding site O1 to G in binding site O2 (FIG. 5).

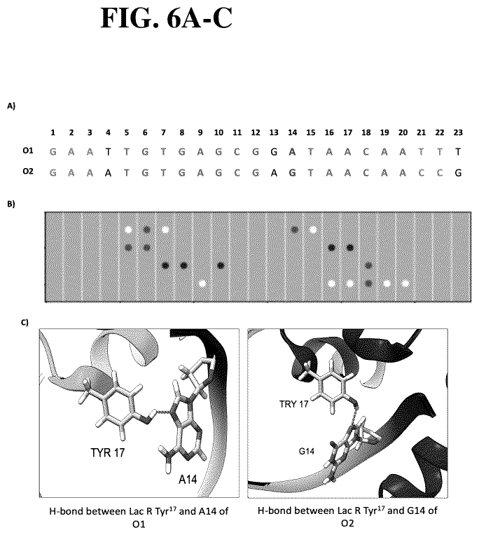

Similarly, the operator O1 was aligned to operator O3 to see which contacts both sequences have in common. Many differences were observed between the O3 sequence relative to O1. However, most of the conserved nucleobases in both operators make the same bonds and contacts with Lac R protein. Additionally, it was observed that there are two hydrogen bonds maintained despite the difference of the nucleobase identity from A-T in O1 to C-G in O3 at location 16 (FIG. 6).

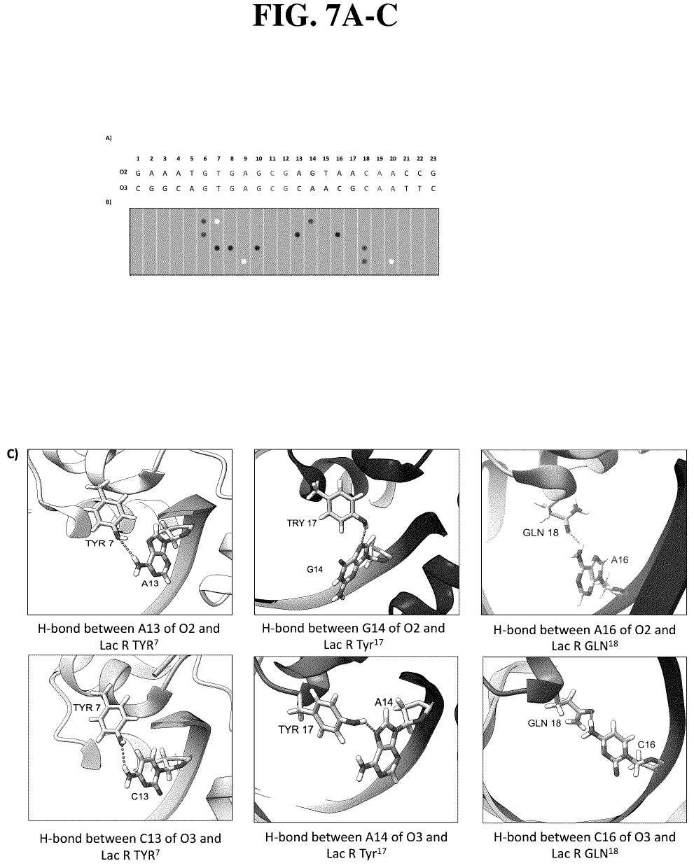

Next, operators O2 and O3 were aligned together. It was found that most of bonds and contacts originate from the conserved base pairs in the two operators. Interestingly, there are three hydrogen bonds maintained in the two operators regardless of the identity of the nucleobases in three different locations: 13, 14 and 16 (FIG. 7).

This present disclosure can shed new light on previous studies that investigated the binding interface of Lac R protein, and its DNA-binding sites. There were four amino acids noted to be responsible for the recognition of target DNA: Arg²², Gln¹⁸, Tyr⁷ and Tyr¹⁷ which agreed with previous studies (FIG. 5-7). The Tyr17 hydroxyl group is responsible for the hydrogen bonding to location 14 in all operators (FIG. 5-7). It was previously observed that Tyr¹⁷ makes hydrogen bond to the 7-position-N in either A or G.

Kalodimos et al. emphasized the importance of Tyr¹⁷ hydroxyl group in the specific binding of Lac R. They showed that mutating Tyr¹⁷ to Phe (Y17F) dropped the affinity ˜100-fold (26). They also showed that the mutant repressor has 10-fold reduction in binding affinity to nonspecific sequences relative to the wild-type repressor. Through the lens of the present data, it is interpreted that this 100-fold affinity reduction has been, in part, due to the protein losing one of the key contacts used to identify its sequence: the Tyr-OH group that contacts G/A at position 14. Even though the base pair changed, the hydrogen bonding pattern was maintained allowing the protein to recognize the site without having to mutate itself. The findings affirm that Lac R could recognize specific distinct patterns of contacts and highlight some interactions that may have been lost to evolutionary analyses made based on the base pair identity.

The Lac R binding a symmetrical sequence was next analyzed. The hydrogen bonds and contacts pattern were verified using the NMR structure of the Lac R protein and this sequence, taken from Spronk et. al. (24) . The Lac R headpiece consists of 3 helices in a canonical helix-turn-helix DNA binding motif plus 9 more residues at the C-terminal that form the so-called hinge region a-helix upon binding to its specific DNA sequence (21). In case of non-specific binding of Lac-R or the absence of the DNA, these 9 residues remain unstructured, which helps in distinguishing the specific binding mode of Lac R from the non-specific binding mode (21, 26). Although this symmetrical sequence is not one of the known Lac R binding sites, Lac R binds to it and forms the hinge region a-helix which used to be seen in the specific binding mode (24, 26).

The symmetrical sequence includes 22 base pairs (Table 1). The first 11 base pairs are identical to the first 11 base pairs of the Lac R binding site O1, but the second half has a different sequence. The binding pattern for the symmetrical sequence was investigated to understand how Lac R could identify and bind it, forming the hinge region, even though it is not one of its known binding sites. For the binding pattern inspection, the published NMR structure by Spronk et al. was used (PDB ID: 1CJG) to verify the hydrogen bonds and contacts for the symmetrical sequence. Then, the binding pattern of the symmetrical sequence was compared to the binding pattern of Lac R indispensable operator O1 since they share the same sequence in the first 11 base pairs.

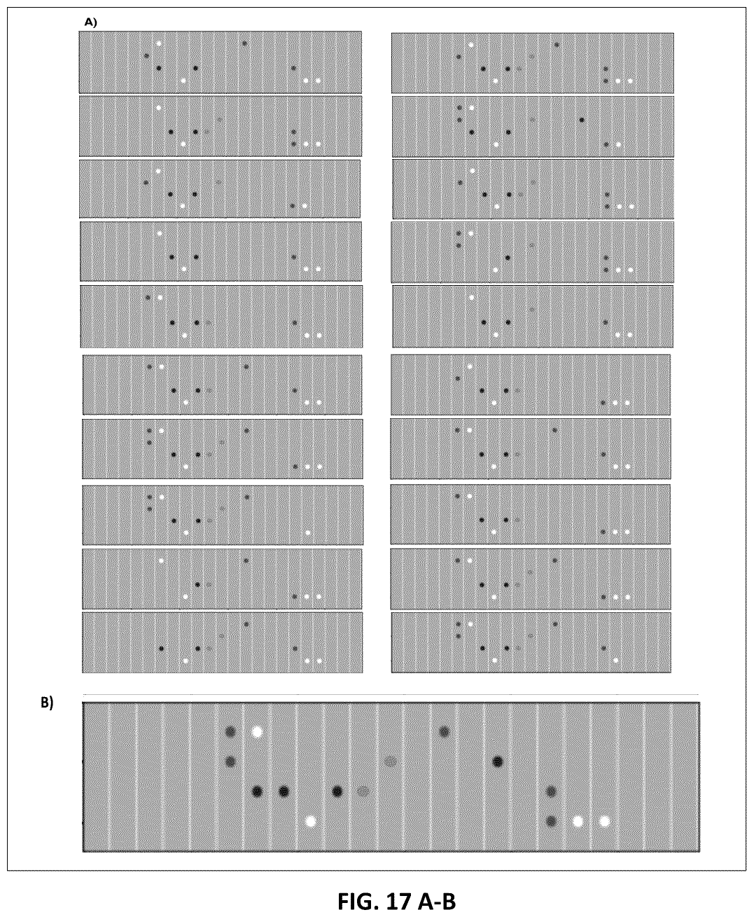

During the alignment, it was noted that O1 operator is longer than the symmetrical sequence by one base pair. Adding a blank space to account for this, the 2 sequences were aligned, and 18 common bonds and contacts were found (FIG. 17). The blank space was entered in position 12 to avoid impacting any area where Lac R should bind. These 18 bonds and contacts represent 60% of the hydrogen bonds and contact, that potentially can be made, shown in O1 which may be enough for the protein to define the symmetrical operator as a binding site and allow formation of the hinge region despite the missing 40% of contacts. It is believed this 60% sequence is the minimal information required and note that the certain position 16 shows hydrogen bonds formed despite changes in base pair identity.

### Controller Protein-DNA Specific Binding Results and Data Analysis

The restriction-modification (RM) system is considered a primitive immune system in bacteria that protects them from bacteriophage infection (10, 27). The proteins that regulate this system are called Controller proteins (10). The operator sequence includes two binding sites: O_{L} binds with a higher affinity, compared to O_{R} (10). Martin et al., showed the crystal structure of C-protein binds O_{R} only as a dimer and O_{L}+O_{R} as a tetramer (27). Surprisingly, C-protein doesn't bind O_{R} with a helix-turn-helix (HTH) motif, it binds ‘end-on’ to O_{R} making very few interactions (27). The protein structure in this complex closely matches the free protein structure. (27) It was also shown that O_{L} binding increases the affinity of C-protein binding at O_{R} by two orders of magnitude by opening the major groove of O_{R} to bind another C-protein dimer (27).

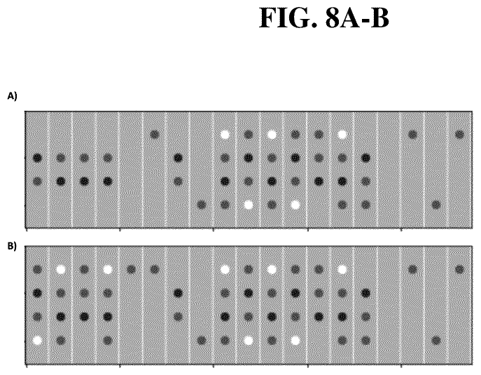

C-protein recognizes three DNA sequences, which were used to make a consensus pattern (FIG. 8A).(10) However, it was considered that C-protein doesn't bind O_{R} independently, it requires O_{L} binding first. Thus, a second consensus pattern of only O_{L} and O_{M} (OLM consensus, FIG. 8B) was made. As predicted, this consensus shows more interactions because the C-protein can identify both operators independently and only two DNA sequences are compared. In the OLM consensus pattern, nucleotide positions are seen where the whole base pair is preserved; ones where hydrogen bonds are preserved but the base pair themselves are different, and ones where nothing is preserved.

The crystal structures of O_{L} and O_{M} were used to refine the hydrogen bonds in the OLM consensus. Four crystal structures are available: two crystal structure for C-protein-O_{L} complex (PDB IDs: 3S8Q and 4IWR)(27-29), one crystal structure for protein-O_{L}+O_{R} complex (PDB ID: 3CLC) (29), and one structure for protein-O_{M} complex (PDB ID: 3UFD) (10).

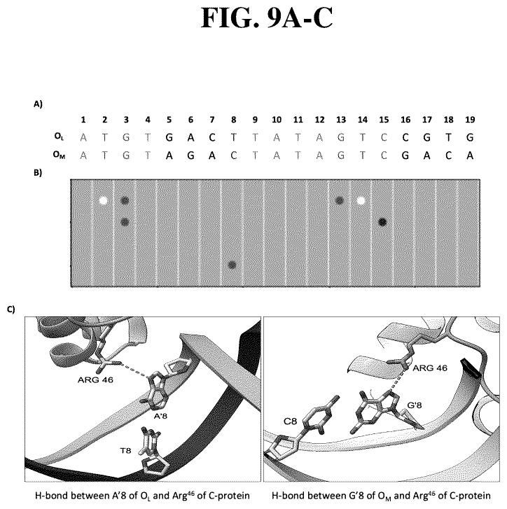

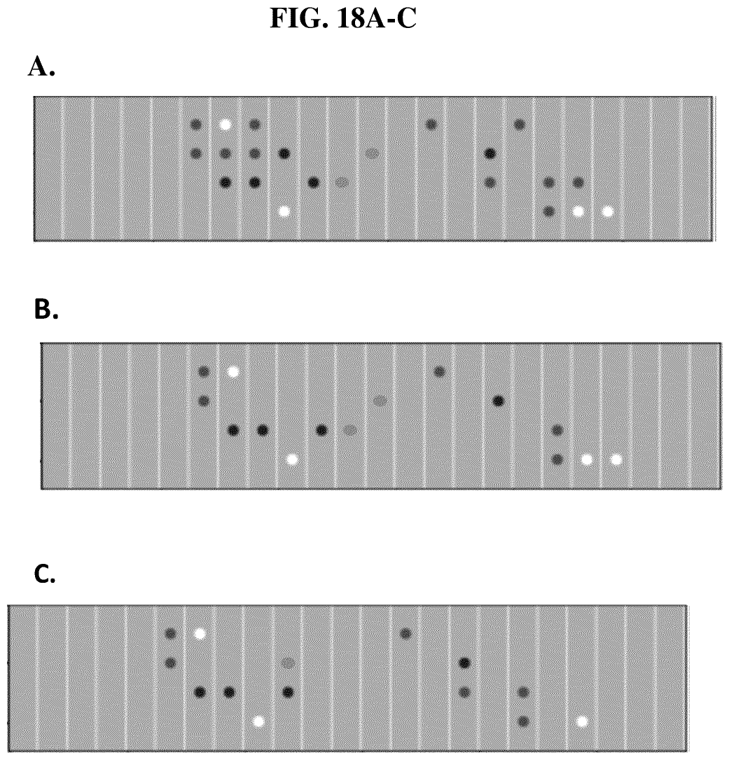

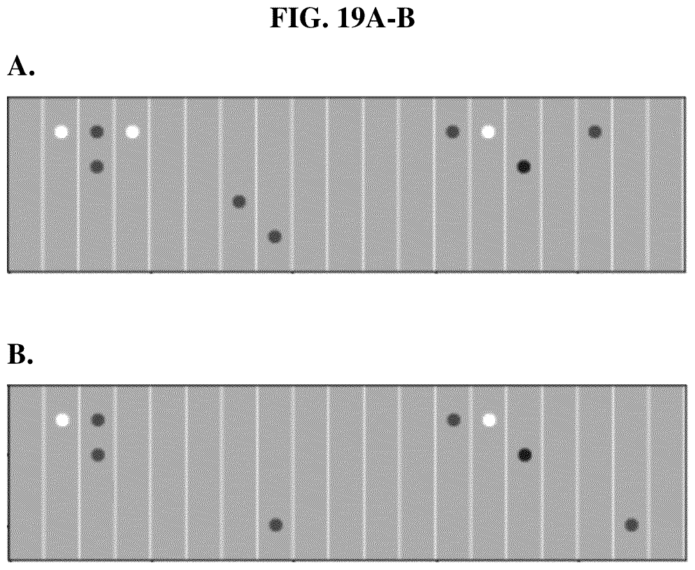

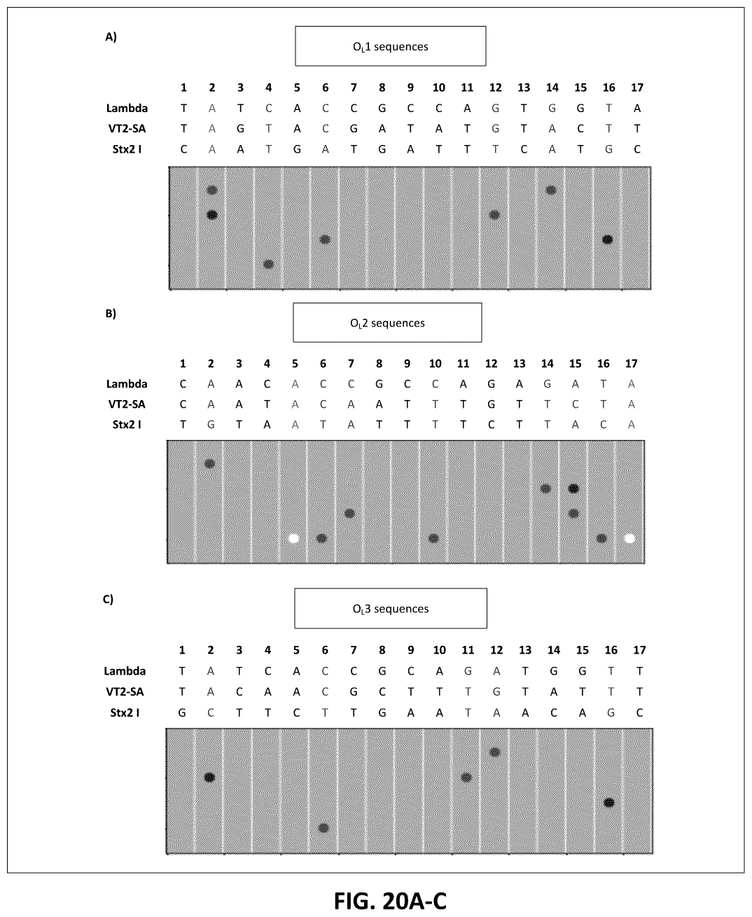

Using the available crystal structures of O_{L}, ten bonds and interactions in the OLM consensus were verified while the available crystal structure of O_{M} only verified eight (FIG. 18). By aligning the two refined patterns together, a distinct pattern of seven bonds and interactions were found (FIG. 9B). Analyzing the base pairs that contribute to this distinct pattern, six bonds and interactions were found that come mainly from conserved base pairs in the two operators and there is one bond in location eight coming from different base pairs in the two operators (T-A and G-C) (FIG. 9A). Interestingly, the center TATA sequence did not contribute directly with specific bonds from the major groove to the specific recognition and binding. It is believed this is due to structural aspects, indirect readout, and other considerations as none of the hydrogen bonds are used in any of the structures.

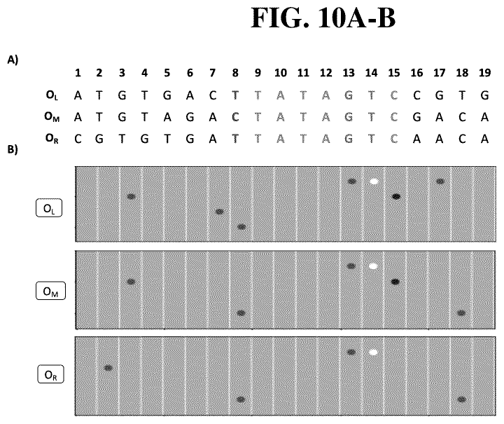

The verified hydrogen bond patterns from the three operators were compared to see how much the binding pattern of O_{R} matches O_{L} and O_{M}. The results showed that O_{R} has a distorted distinct pattern compared to the other two operators. However, the hydrogen bond in location 8 that comes from different base pairs is maintained in the low affinity O_{R} (FIG. 10). It is believed this distorted pattern is what contributes to the lower affinity, O_{L} binding is thus required to help position the C-protein and assist in deforming the DNA to properly form the required contacts for DNA binding.

### λ-Phage Repressor-DNA Specific Binding Results and Data Analysis

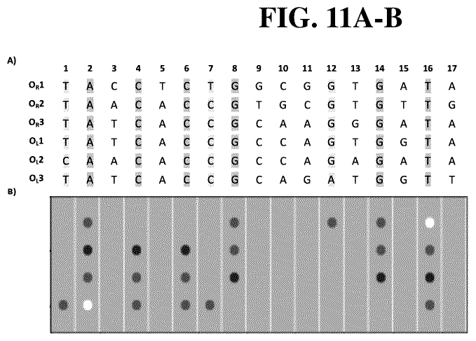

Bacteriophage λ is a virus that infects E. coli. Upon infection the phage can enter into either a silent life cycle or a virulent life cycle (30). This decision is, in part, controlled by a transcriptional repressor protein named CI (31, 32). CI binds in two different promoter regions of the phage genome P_{R} and P_{L} (32). Each of these promoters comprises three different operator sites where CI binds as a dimer (31, 32). The six operators are termed as O_{R}1, O_{R}2, O_{R}3, O_{L}1, O_{L}2, and O_{L}3. The genomic sequences of λ-phage's six operator sites are available online in the NCBI taxonomy database (33, 34). The consensus pattern was made using the six operator sequences (FIG. 11B). There are three positions where hydrogen bonding is preserved despite variation in the base pair identity, six positions where the nucleotides are preserved, and eight positions where nothing is preserved (FIG. 11A).

The published crystal structure for CI-O_{L}1 complex from Beamer and Pabo (1LMB) was used for the analysis due to its high resolution (35). Since there are no crystal structures available for the other protein-operator complexes, the DNA sequence of 1LMB in UCSF Chimera was mutated to the other five operator sequences. The complex structures were minimized to relax the mutated DNA complexes before detecting the hydrogen bonds. The λ-CI protein has a flexible arm that interacts with specific DNA nucleobases (11). It was noticed that this arm is cut off in one of the protein monomers. Therefore, the sequence of each of the six operators were mutated into the crystal structure twice, once running forward and one running in the reverse direction such that each monomer is contacting the DNA close to the 5′ and 3′ end of the same strand. This allows one to approximate the interaction between the flexible arm and all of the nucleotides.

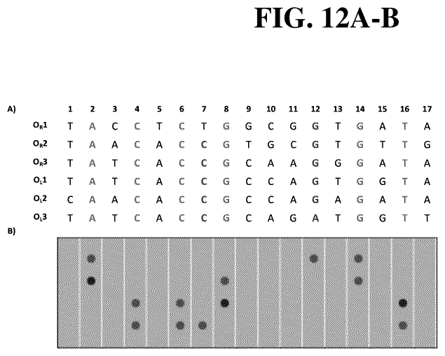

Then, all the DNA-protein complexes were prepared, and the hydrogen bonds were verified to generate the refined distinct pattern, showing what information is maintained among the six sequences (FIG. 12B). It was found that most of the hydrogen bonds are from base pairs that were conserved in the six operators except for two hydrogen bonds at locations 7 and 12 (FIG. 12B).

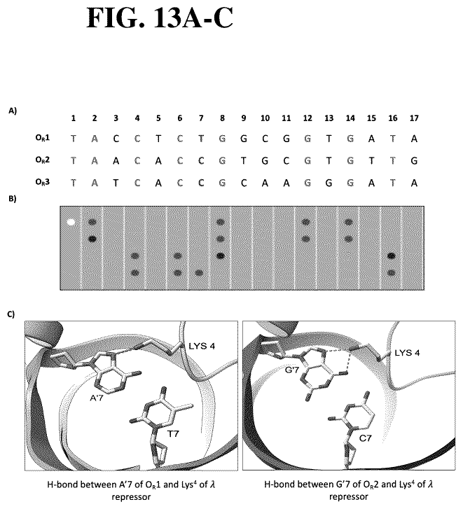

The individual left and right operators were then aligned to investigate how CI can tell them apart. The first alignment was for the three binding sites of the right operator (O_{R}), and it showed that most of the hydrogen bonds shown in the distinct pattern are from conserved base pairs except for hydrogen bonds at location 7 which are maintained despite the change of the base pair identity (FIG. 13).

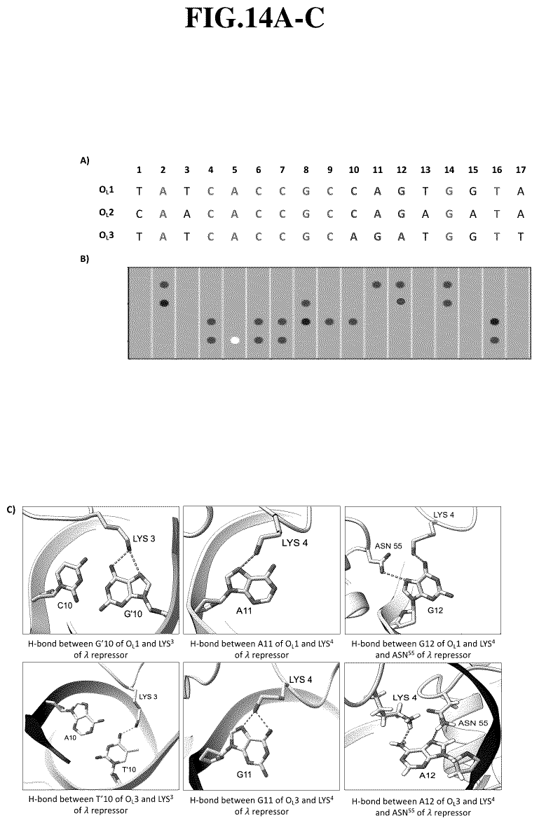

In the next step, it was tested to see if the left and right operators have unique information that assists the CI protein in recognizing one set of sequences over the other. The binding sites from the left operators were aligned together. Most of the base pairs are conserved and show the same pattern of hydrogen bonding. However, three non-conserved base pairs show the same pattern of hydrogen bonding at locations 10, 11, and 12 (FIG. 14). In addition, it was observed that the amino group of Lys⁴ is unexpectedly donating a hydrogen bond to the N atom of 6-position of adenine 12 in O_{L}3 (FIG. 14C) (36).

### The Alignment of λ-Phage'S Binding Sites With Other Strains' Binding Sites

λ-phage is one strain of lambdoid phages known to produce Shiga toxins (37). To better understand how information transfers through evolution, a comparative analysis of the λ phage's binding sites and the binding sites of other evolutionary related phages was run. Enterobacteria phage VT2-Sakai (VT2-SA) and Stx2 converting phage I (Stx2 I) were included due to their sequence availability, close evolutionary relation, and the fact that they produce Shiga toxins. Each strain has six binding sites, the same as λ phage. Six alignments were run, one for each operator site for each of the three strains. For the verification step, the contacts from λ-phage were used as the other two strains do not have published structures of CI bound to DNA. The bonds verified from the λ-phage crystal structure ILMB, were kept while the other bonds were removed.

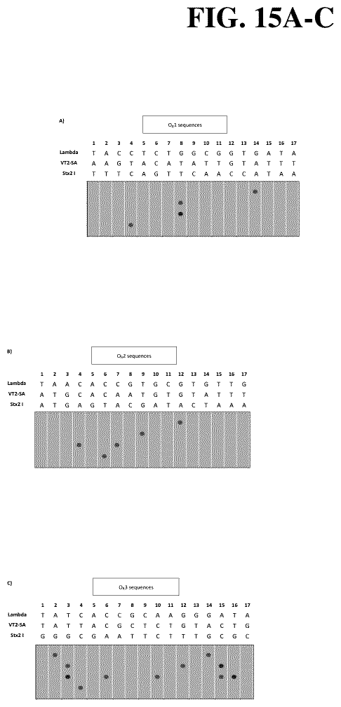

From this analysis it was found that almost all the hydrogen bonds conserved between the three strains arise from different nucleotides (FIG. 15). These results reveal some information is hidden if the nucleobase identity is only considered in a comparative analysis. Interestingly, the O_{R}1 sequence had the least amount of overlap between phage strains, but is noted to have the highest affinity (38). It is believed that a more selective O_{R}1 binding allows the phage to screen for its own DNA from co-infecting phages in the same bacterium.

Protein-DNA binding is vital, underpinning many biological processes such as replication, transcription, and more in all known organisms. Thus, understanding how DNA-binding proteins recognize and bind specifically to their target DNA can contribute to the development of new gene therapies and drugs.

Many factors that contribute to specific recognition and binding are represented in direct and indirect readout of DNA by the protein. Most studies agreed on direct readout as the main factor for recognition and specificity with consideration to the other indirect readout factors. In this work, an effort was made to address elements of direct readout, namely the hydrogen bond pattern exposed to proteins in the major groove.

In this work, a class of hydrogen bonds and van der Waals interactions were studied that may be overlooked with standard alignment methods and developed an algorithm that can extract them from sequence information. This study had a specific focus on those DNA-binding proteins that can recognize and bind more than one sequence. The belief is that each specific protein binds its corresponding DNA sequences through a network of hydrogen bonds and contacts in the major groove, and analyses focused on the base pair identity may overlook key interactions. The study comprised three proteins that are known of multiple binding sites, Lac R, C-protein, and λ-CI. The different DNA sequences of each protein were analyzed through the designed algorithm to extract the hydrogen bonds and non-covalent contacts maintained in these different sequences to reveal any overlooked key interactions.

From the studies disclosed herein, many of these key interaction bonds were highlighted. All the examples used in this study have positions where DNA base pairs are variable, but the hydrogen bonds that connect the protein with DNA are maintained. Interestingly, in Lac R and C-protein, some conserved nucleotides didn't contribute to the network of hydrogen bonds as was expected. These may take part in indirect readout or other structural aspects of DNA recognition which is beyond the scope of this work.

To conduct the experiments, it was fortunate that published data exists that could be used to evaluate whether or not a protein can recognize a different sequence that maintains the same hydrogen bond pattern. A symmetrical sequence binding to Lac-R was chosen for this analysis (24). Interestingly, Lac-R could recognize and specifically bind this symmetrical sequence, forming the hinge region, although it retains 60% of the contacts that potentially made by Lac R-O1. On the other side, Lac R couldn't show specific binding to a sequence that doesn't maintain the same hydrogen bond pattern and instead it bound non-specifically without forming the hinge region (26) which further supports the belief that DNA-binding proteins recognize their DNA target through a network of hydrogen bonds and contacts in the major groove and analyses of base pair identity may overlook some important key interactions for recognition and specificity.

Similarly, the experimental data disclosed herein adds a new perspective to the work of Lin and Guo. Their paper showed that certain proteins only read information from one strand of DNA. In those situations, the effect of maintaining a hydrogen bond can further reduce specificity. A to G mutations maintain the 7-position nitrogen, therefore proteins making that contact could not screen these two nucleotides from one-another based solely on the 7-position lone pair. That leaves only one hydrogen bond available to discern the sequence (the 6-position amino or carbonyl group). It is shown that the information can be even more variable in that case thereby lessening their specificity more.

Some possibly new evolutionary relationships are shown between different phage strains and ways that viruses can screen genomes to bind the correct operator site. The algorithm disclosed herein indicated the presence of hydrogen bonds that are shared among the binding sites of the three strains. The consideration of the hydrogen bond pattern presented by the nucleobase in the analysis revealed some hidden information which might be ignored when considering only the base pair identity. It is possible that this information may have a hand in the evolutionary trajectory of phages. Based on the observed results, it is suspected that if an operator site mutates, the CI protein will have to mutate accordingly to regain proper binding affinity. However, if the mutation does not change the information (as described here) then no CI mutations would be required. Thus, it is possible that some mutations are benign and allow for other mutations elsewhere to accumulate. In addition, it is suspected that the CI repressor of λ-phage might bind the operator sites of either VT2-SA or Stx2 I with fair affinity.

From this study, it is found that the most common nucleotide change that maintains hydrogen bonds comes from purine to purine. In this case, the 7-position nitrogen provides a lone pair of electrons for hydrogen bonding. This is responsible for the majority of the flexibility seen and is a common target for DNA-binding proteins. Adenine to Cytosine mutations are also seen to retain a hydrogen bond donor from the amino group of Adenine or Cytosine and one hydrogen bond acceptor from the Carbonyl group on either Thymine or Guanine on the complement DNA strand. These combinations provide a lot of information-retention when DNA is mutating. Each base pair has a mutant that can retain the hydrogen bonding character. These interactions are often overlooked if one is only considering the identity of the base pairs themselves.

It was noted that the change in the nucleobases is not limited to the typical change between the purine bases (A and G) or the pyrimidine bases (C and T), but also it happens to be a change from purine base to pyrimidine base and vice versa.

Although each of the three proteins, Lac R, C-protein, and λ-CI, could recognize and specifically bind to multiple binding sites, it is believed that the changes in the base pairs among these different binding sites are responsible for the variation of its affinity of binding that was discussed above in each protein's respective results section. Also, the variation of the base pairs from G-C to A-T could affect the structure of the DNA which, in part, contributed to the different binding affinities among the operators of the three proteins.

From the results disclosed herein it can be concluded that DNA-binding proteins recognize their DNA target through a network of hydrogen bonds and contacts in the major groove. The focus solely on the identity of the nucleobases can lead analyses to overlook some important key interactions for recognition and specificity. These observations will have a multitude of applications. For example, protein design groups seeking to develop artificial transcription factors (ATFs) could use the methods disclosed herein to better screen out the minimal required information and target those hydrogen bond partners when looking at the interface. This could lead to ATFs with specificity toward multiple sequences as well as a deeper understanding of how existing ones recognize their target DNA. Similarly, structural biologists will benefit from this work by better identifying hydrogen bonds that could be made between proteins and their corresponding DNA-binding sites.

Those studying evolution will also benefit from this new type of analysis as it seeks to better identify the information itself within the DNA. Focusing on this can help researchers trace how certain mutations can arise first and why some mutations cause more noticeable effects than others. As discussed above, the methods disclosed herein can help those groups identify which pieces of the information displayed are more or less important, and from there how interactions with different proteins can be more or less affected by evolutionary changes.

### REFERENCES

1. Lin, M. and Guo, J. (2019) New insights into protein-DNA binding specificity from hydrogen bond based comparative study. Nucleic Acids Res., 47, 11103-11113.
2. Emamjomeh, A., Choobineh, D., Hajieghrari, B., MahdiNezhad, N. and Khodavirdipour, A. (2019) DNA-protein interaction: identification, prediction and data analysis. Mol. Biol. Rep., 46, 3571-3596.
3. Luscombe, N. M. (2001) Amino acid-base interactions: a three-dimensional analysis of protein-DNA interactions at an atomic level. Nucleic Acids Res., 29, 2860-2874.
4. Garvie, C. W. and Wolberger, C. (2001) Recognition of Specific DNA Sequences. Mol. Cell, 8, 937-946.
5. Lin, M. and Guo, J. (2019) New insights into protein-DNA binding specificity from hydrogen bond based comparative study. Nucleic Acids Res., 47, 11103-11113.
6. Sarai, A. and Kono, H. (2005) Protein-DNA Recognition Patterns and Predictions. Annu. Rev. Biophys. Biomol. Struct., 34, 379-398.
7. Rohs, R., Jin, X., West, S. M., Joshi, R., Honig, B. and Mann, R. S. (2010) Origins of Specificity in Protein-DNA Recognition. Annu. Rev. Biochem., 79, 233-269.
8. Jayaram, B. and Jain, T. (2004) The Role of Water in Protein-DNA Recognition. Annu. Rev. Biophys. Biomol. Struct., 33, 343-361.
9. Lejeune, D., Delsaux, N., Charloteaux, B., Thomas, A. and Brasseur, R. (2005) Protein-nucleic acid recognition: Statistical analysis of atomic interactions and influence of DNA structure. Proteins Struct. Funct. Bioinforma., 61, 258-271.
10. Ball, N. J., McGeehan, J. E., Streeter, S. D., Thresh, S.-J. and Kneale, G. G. (2012) The structural basis of differential DNA sequence recognition by restriction-modification controller proteins. Nucleic Acids Res., 40, 10532-10542.
11. Hochschild, A., Douhan, J. and Ptashne, M. (1986) How λ repressor and λ Cro distinguish between OR1 and OR3. Cell, 47, 807-816.
12. Kumar, S., Bhardwaj, V. K., Singh, R., Das, P. and Purohit, R. (2022) Identification of acridinedione scaffolds as potential inhibitor of DENV-2 C protein: An in silico strategy to combat dengue. J. Cell. Biochem., 123, 935-946.
13. Rajendran, V., Purohit, R. and Sethumadhavan, R. (2012) In silico investigation of molecular mechanism of laminopathy caused by a point mutation (R482W) in lamin A/C protein. Amino Acids, 43, 603-615.
14. Bhardwaj, V. K., Oakley, A. and Purohit, R. (2022) Mechanistic behavior and subtle key events during DNA clamp opening and closing in T4 bacteriophage. Int. J. Biol. Macromol., 208, 11-19.
15. Pettersen, E. F., Goddard, T. D., Huang, C. C., Couch, G. S., Greenblatt, D. M., Meng, E. C. and Ferrin, T. E. (2004) UCSF Chimera?A visualization system for exploratory research and analysis. J. Comput. Chem., 25, 1605-1612.
16. Pettersen, E. F., Goddard, T. D., Huang, C. C., Meng, E. C., Couch, G. S., Croll, T. I., Morris, J. H. and Ferrin, T. E. (2021) UCSF ChimeraX : Structure visualization for researchers, educators, and developers. Protein Sci., 30, 70-82.
17. Goddard, T. D., Huang, C. C., Meng, E. C., Pettersen, E. F., Couch, G. S., Morris, J. H. and Ferrin, T. E. (2018) UCSF ChimeraX: Meeting modern challenges in visualization and analysis: UCSF ChimeraX Visualization System. Protein Sci., 27, 14-25.
18. Harris, C. R., Millman, K. J., van der Walt, S. J., Gommers, R., Virtanen, P., Cournapeau, D., Wieser, E., Taylor, J., Berg, S., Smith, N. J., et al. (2020) Array programming with NumPy. Nature, 585, 357-362.
19. Caswell, T. A., Droettboom, M., Hunter, J., Lee, A., Firing, E., Stansby, D., Klymak, J., Andrade, E. S. D., Nielsen, J. H., Varoquaux, N., et al. (2019) matplotlib/matplotlib: REL: v3.1.1. 10.5281/ZENODO.3264781.
20. Mills, J. E. J. and Dean, P. M. (1996) Three-dimensional hydrogen-bond geometry and probability information from a crystal survey. J. Comput. Aided Mol. Des., 10, 607-622.
21. Romanuka, J., Folkers, G. E., Biris, N., Tishchenko, E., Wienk, H., Bonvin, A. M. J. J., Kaptein, R. and Boelens, R. (2009) Specificity and Affinity of Lac Repressor for the Auxiliary Operators O2 and O3 Are Explained by the Structures of Their Protein-DNA Complexes. J. Mol. Biol., 390, 478-489.
22. Kalodimos, C. G., Boelens, R. and Kaptein, R. (2004) Toward an Integrated Model of Protein-DNA Recognition as Inferred from NMR Studies on the L ac Repressor System. Chem. Rev., 104, 3567-3586.
23. Kopke Salinas, R., Folkers, G. E., Bonvin, A. M. J. J., Das, D., Boelens, R. and Kaptein, R. (2005) Altered Specificity in DNA Binding by the lac Repressor: A Mutant lac Headpiece that Mimics the gal Repressor. ChemBioChem, 6, 1628-1637.
24. Spronk, C. A., Bonvin, A. M., Radha, P. K., Melacini, G., Boelens, R. and Kaptein, R. (1999) The solution structure of Lac repressor headpiece 62 complexed to a symmetrical lac operator. Structure, 7, 1483-S3.
25. Kalodimos, C. G. (2002) Plasticity in protein-DNA recognition: lac repressor interacts with its natural operator O1 through alternative conformations of its DNA-binding domain. EMBO J., 21, 2866-2876.
26. Kalodimos, C. G., Biris, N., Bonvin, A. M. J. J., Levandoski, M. M., Guennuegues, M., Boelens, R. and Kaptein, R. (2004) Structure and Flexibility Adaptation in Nonspecific and Specific Protein-DNA Complexes. Science, 305, 386-389.
27. Martin, R. N. A., McGeehan, J. E., Ball, N. J., Streeter, S. D., Thresh, S.-J. and Kneale, G. G. (2013) Structural analysis of DNA-protein complexes regulating the restriction-modification system Esp 1396I. Acta Crystallograph. Sect. F Struct. Biol. Cryst. Commun., 69, 962-966.
28. McGeehan, J. E., Ball, N. J., Streeter, S. D., Thresh, S.-J. and Kneale, G. G. (2012) Recognition of dual symmetry by the controller protein C.Esp1396I based on the structure of the transcriptional activation complex. Nucleic Acids Res., 40, 4158-4167.
29. McGeehan, J. E., Streeter, S. D., Thresh, S.-J., Ball, N., Ravelli, R. B. G. and Kneale, G. G. (2008) Structural analysis of the genetic switch that regulates the expression of restriction-modification genes. Nucleic Acids Res., 36, 4778-4787.
30. Salmond, G. P. C. and Fineran, P. C. (2015) A century of the phage: past, present and future. Nat. Rev. Microbiol., 13, 777-786.
31. Stayrook, S., Jaru-Ampornpan, P., Ni, J., Hochschild, A. and Lewis, M. (2008) Crystal structure of the λ repressor and a model for pairwise cooperative operator binding. Nature, 452, 1022-1025.
32. Gao, N., Shearwin, K., Mack, J., Finzi, L. and Dunlap, D. (2013) Purification of bacteriophage lambda repressor. Protein Expr. Purif., 91, 30-36.
33. Schoch, C. L., Ciufo, S., Domrachev, M., Hotton, C. L., Kannan, S., Khovanskaya, R., Leipe, D., Mcveigh, R., O'Neill, K., Robbertse, B., et al. (2020) NCBI Taxonomy: a comprehensive update on curation, resources and tools. Database, 2020, baaa062.
34. Sayers, E. W., Cavanaugh, M., Clark, K., Ostell ,J., Pruitt, K. D. and Karsch-Mizrachi, I. (2019) GenBank. Nucleic Acids Res., 47, D94-D99.
35. Beamer, L. J. and Pabo, C. O. (1992) Refined 1.8 Å crystal structure of the λ repressor-operator complex. J. Mol. Biol., 227, 177-196.
36. Kagra, D., Prabhakar, P. S., Sharma, K. D. and Sharma, P. (2020) Structural Patterns and Stabilities of Hydrogen-Bonded Pairs Involving Ribonucleotide Bases and Arginine, Glutamic Acid, or Glutamine Residues of Proteins from Quantum Mechanical Calculations. ACS Omega, 5, 3612-3623.
37. Fattah, K. R., Mizutani, S., Fattah, F. J., Matsushiro, A. and Sugino, Y. (2000) A comparative study of the immunity region of lambdoid phages including Shiga-toxin-converting phages. Molecular basis for cross immunity. Genes Genet. Syst., 75, 223-232.
38. Bell, C. E., Frescura, P., Hochschild, A. and Lewis, M. (2000) Crystal Structure of the 2 Repressor C-Terminal Domain Provides a Model for Cooperative Operator Binding. Cell, 101, 801-811.

TABLE 2
Table 1. The sequences of the binding sites.

| DNA- | | | | |
|---|---|---|---|---|
| Binding | | SEQ ID | | |
| Protein | Operator | NO. | DNA sequence | PDB ID |
| Lac repressor | O1 | 1 | GAATTGTGAGCGGATAACAATTT | 2KEI, 1L1M |
| | O2 | 2 | GAAATGTGAGCGAGTAACAACCG | 2KEJ |
| | O3 | 3 | CGGCAGTGAGCGCAACGCAATTC | 2KEK |
| | Symmetrical | 4 | GAATTGTGAGCGCTCACAATTC | 1CJG |
| | sequence | | | |
| C-protein | O_{L} | 5 | ATGTGACTTATAGTCCGTG | 3S8Q, 4IWR |
| | O_{R} | 6 | CGTGTGATTATAGTCAACA | 3CLC |
| | O_{M} | 7 | ATGTAGACTATAGTCGACA | 3UFD |
| λ-repressor | O_{R}1 | 8 | TACCTCTGGCGGTGATA | 1LMB |
| | | | | (mutated) |
| | O_{R}2 | 9 | TAACACCGTGCGTGTTG | 1LMB |
| | | | | (mutated) |
| | O_{R}3 | 10 | TATCACCGCAAGGGATA | 1LMB |
| | | | | (mutated) |
| | O_{L}1 | 11 | TATCACCGCCAGTGGTA | 1LMB |
| | O_{L}2 | 12 | CAACACCGCCAGAGATA | 1LMB |
| | | | | (mutated) |
| | O_{L}3 | 13 | TATCACCGCAGATGGTT | 1LMB |
| | | | | (mutated) |

What is claimed:

1. A method for identifying shared information between a DNA sequence and their DNA binding partner comprising the steps of:
(i) obtaining publicly available the DNA sequences of corresponding DNA binding sites for the given DNA binding protein of interest for input into an algorithm to generate hydrogen bond patterns, the algorithm configured to map base pairs of the DNA sequences to preconfigured patterns, each of the preconfigured patterns comprising at least three of: an indication of a hydrogen bond donor (“HBD”), an indication of a hydrogen bond acceptor (“HBA”), an indication of a thymine methyl group (“TMG”), or an indication of none; (ii) converting the individual base pairs into a four-slot vertical array of designated hydrogen bond donors, acceptors, methyl groups or if nothing is in that position;
(iii) aligning the hydrogen bond patterns to obtain one consensus pattern that is shared among all the protein binding sites;
(iv) obtaining the crystal or NMR structures for the various protein-DNA complexes from the publicly available Protein Data Bank and verifying through the crystal and NMR structures that the maintained bonds in the alignment are indeed used by the protein for binding and recognition; and
(v) obtaining the final refined patterns by aligning the verified contacts that were detected in the published structures for each binding site complex.

2. The method of claim 1, wherein the algorithm is configured to:
assign, to an Adenine-Thymine base pair, a preconfigured pattern of HBA, HBD, HBA, TMG;
assign, to a Thymine-Adenine base pair, a preconfigured pattern of TMG, HBA, HBD, HBA;
assign, to a Cytosine-Guanine base pair, a preconfigured pattern of None, HBD, HBA, HBA;
assign, to a Guanine-Cytosine base pair, a preconfigured pattern of HBA, HBA, HBD, None.

3. The method of claim 1, wherein step (iv) is conducted using USFC Chimera X.

4. The method of claim 1, wherein the DNA binding site is a sequence that regulates transcription.

5. The method of claim 1, wherein the DNA binding protein of interest is a transcription factor. regulates transcription.

6. The method of claim 5, wherein the transcription factor is an activator or repressor.

7. The method of claim 1, wherein the DNA-binding protein is a nuclease.

8. A method is provided for determining the minimum DNA sequence and their DNA binding protein of interest, comprising the steps of:
(i) obtaining publicly available DNA sequences corresponding to DNA binding sites for the given DNA binding protein of interest for input into an algorithm to generate hydrogen bond patterns, the algorithm configured to map base pairs of the DNA sequences to preconfigured patterns, each of the preconfigured patterns comprising at least three of: an indication of a hydrogen bond donor (“HBD”), an indication of a hydrogen bond acceptor (“HBA”), an indication of a thymine methyl group (“TMG”), or an indication of none;
(ii) converting the individual base pairs into a four-slot vertical array of designated hydrogen bond donors, acceptors, methyl groups or if nothing is in that position;
(iii) aligning the hydrogen bond patterns to obtain one consensus pattern that is shared among all the protein binding sites;
(iv) obtaining the crystal or NMR structures for the various protein-DNA complexes from the publicly available Protein Data Bank and verifying through the crystal and NMR structures that the maintained bonds in the alignment are indeed used by the protein for binding and recognition; and
(v) obtaining the final refined patterns by aligning the verified contacts that were detected in the published structures for each binding site complex thereby providing the shared information between the DNA sequence and their DNA binding partner of interest.

9. The method of claim 8, wherein the algorithm is configured to:
assign, to an Adenine-Thymine base pair, a preconfigured pattern of HBA, HBD, HBA, TMG;
assign, to a Thymine-Adenine base pair, a preconfigured pattern of TMG, HBA, HBD, HBA;
assign, to a Cytosine-Guanine base pair, a preconfigured pattern of None, HBD, HBA, HBA;
assign, to a Guanine-Cytosine base pair, a preconfigured pattern of HBA, HBA, HBD, None.

10. The method of claim 8, wherein step (iv) is conducted using USFC Chimera X.

11. The method of claim 8, wherein the DNA binding site is a sequence that regulates transcription.

12. The method of claim 8, wherein the DNA binding protein of interest is a transcription factor. regulates transcription.

13. The method of claim 12, wherein the transcription factor is an activator or repressor.

14. The method of claim 8, wherein the DNA-binding protein is a nuclease.

15. A method for identifying an engineered DNA-binding protein that binds to a target DNA sequence of interest, said method comprises the steps of : (i) obtaining publicly available target DNA sequences of interest corresponding to DNA binding sites for the engineered DNA binding protein for input into an algorithm to generate hydrogen bond patterns, the algorithm configured to map base pairs of the DNA sequences to preconfigured patterns, each of the preconfigured patterns comprising at least three of: an indication of a hydrogen bond donor (“HBD”), an indication of a hydrogen bond acceptor (“HBA”), an indication of a thymine methyl group (“TMG”), or an indication of none; (ii) converting the individual base pairs into a four-slot vertical array of designated hydrogen bond donors, acceptors, methyl groups or if nothing is in that position; (iii) aligning the hydrogen bond patterns to obtain one consensus pattern that is shared among all the protein binding sites; (iv) obtaining the crystal or NMR structures for the protein-DNA complexes and verifying through the crystal and NMR structures that the maintained bonds in the alignment are indeed used by the protein for binding and recognition; and (v) obtaining the final refined patterns by aligning the verified contacts that were detected in the published structures for each binding site complex, thereby identifying an engineered DNA-binding protein that binds to a target DNA sequence of interest.

16. The method of claim 15, wherein the algorithm is configured to:
assign, to an Adenine-Thymine base pair, a preconfigured pattern of HBA, HBD, HBA, TMG;
assign, to a Thymine-Adenine base pair, a preconfigured pattern of TMG, HBA, HBD, HBA;
assign, to a Cytosine-Guanine base pair, a preconfigured pattern of None, HBD, HBA, HBA;
assign, to a Guanine-Cytosine base pair, a preconfigured pattern of HBA, HBA, HBD, None.

17. The method of claim 15, wherein step (iv) is conducted using USFC Chimera X.

18. The method of claim 15, wherein the DNA binding site is a sequence that regulates transcription.

19. The method of claim 15, wherein the DNA binding protein of interest is a transcription factor. regulates transcription.

20. The method of claim 15, wherein the DNA-binding protein is a nuclease.


SEQUENCE LISTING (26 sequences)

SEQ ID NO: 1 - DNA, 23 bp, synthetic construct
  misc_feature 1..23: note = Synthetic: Lac repressor O1
  source 1..23: mol_type = other DNA
gaattgtgagcggataacaattt

SEQ ID NO: 2 - DNA, 23 bp, synthetic construct
  misc_feature 1..23: note = Synthetic: Lac repressor O2
  source 1..23: mol_type = other DNA
gaaatgtgagcgagtaacaaccg

SEQ ID NO: 3 - DNA, 23 bp, synthetic construct
  misc_feature 1..23: note = Synthetic: Lac repressor O3
  source 1..23: mol_type = other DNA
cggcagtgagcgcaacgcaattc

SEQ ID NO: 4 - DNA, 22 bp, synthetic construct
  misc_feature 1..22: note = Synthetic: Symmetrical sequence
  source 1..22: mol_type = other DNA
gaattgtgagcgctcacaattc

SEQ ID NO: 5 - DNA, 19 bp, synthetic construct
  misc_feature 1..19: note = Synthetic: C-protein OL
  source 1..19: mol_type = other DNA
atgtgacttatagtccgtg

SEQ ID NO: 6 - DNA, 19 bp, synthetic construct
  misc_feature 1..19: note = Synthetic: C-protein OR
  source 1..19: mol_type = other DNA
cgtgtgattatagtcaaca

SEQ ID NO: 7 - DNA, 19 bp, synthetic construct
  misc_feature 1..19: note = Synthetic: C-protein OM
  source 1..19: mol_type = other DNA
atgtagactatagtcgaca

SEQ ID NO: 8 - DNA, 17 bp, synthetic construct
  misc_feature 1..17: note = Synthetic: A-repressor OR1
  source 1..17: mol_type = other DNA
tacctctggcggtgata

SEQ ID NO: 9 - DNA, 17 bp, synthetic construct
  misc_feature 1..17: note = Synthetic: A-repressor OR2
  source 1..17: mol_type = other DNA
taacaccgtgcgtgttg

SEQ ID NO: 10 - DNA, 17 bp, synthetic construct
  misc_feature 1..17: note = Synthetic: A-repressor OR3
  source 1..17: mol_type = other DNA
tatcaccgcaagggata

SEQ ID NO: 11 - DNA, 17 bp, synthetic construct
  misc_feature 1..17: note = Synthetic: A-repressor OL1
  source 1..17: mol_type = other DNA
tatcaccgccagtggta

SEQ ID NO: 12 - DNA, 17 bp, synthetic construct
  misc_feature 1..17: note = Synthetic: A-repressor OL2
  source 1..17: mol_type = other DNA
caacaccgccagagata

SEQ ID NO: 13 - DNA, 17 bp, synthetic construct
  misc_feature 1..17: note = Synthetic: A-repressor OL3
  source 1..17: mol_type = other DNA
tatcaccgcagatggtt

SEQ ID NO: 14 - DNA, 17 bp, synthetic construct
  misc_feature 1..17: note = Synthetic: VT2-SA OR1
  source 1..17: mol_type = other DNA
aagtacatattgtattt

SEQ ID NO: 15 - DNA, 17 bp, synthetic construct
  misc_feature 1..17: note = Synthetic: Stx2 I OR1
  source 1..17: mol_type = other DNA
tttcagttcaaccataa

SEQ ID NO: 16 - DNA, 17 bp, synthetic construct
  misc_feature 1..17: note = Synthetic: VT2-SA OR2
  source 1..17: mol_type = other DNA
atgcacaatgtgtattt

SEQ ID NO: 17 - DNA, 17 bp, synthetic construct
  misc_feature 1..17: note = Synthetic: Stx2 I OR2
  source 1..17: mol_type = other DNA
atgagtacgatactaaa

SEQ ID NO: 18 - DNA, 17 bp, synthetic construct
  misc_feature 1..17: note = Synthetic: VT2-SA OR3
  source 1..17: mol_type = other DNA
tattacgctctgtactg

SEQ ID NO: 19 - DNA, 17 bp, synthetic construct
  misc_feature 1..17: note = Synthetic: Stx2 I OR3
  source 1..17: mol_type = other DNA
gggcgaattctttgcgc

SEQ ID NO: 20 - DNA, 17 bp, synthetic construct
  misc_feature 1..17: note = Synthetic: VT2-SA OL1
  source 1..17: mol_type = other DNA
tagtacgatatgtactt

SEQ ID NO: 21 - DNA, 17 bp, synthetic construct
  misc_feature 1..17: note = Synthetic: Stx2 I OL1
  source 1..17: mol_type = other DNA
caatgatgatttcatgc

SEQ ID NO: 22 - DNA, 17 bp, synthetic construct
  misc_feature 1..17: note = Synthetic: VT2-SA OL2
  source 1..17: mol_type = other DNA
caatacaatttgttcta

SEQ ID NO: 23 - DNA, 17 bp, synthetic construct
  misc_feature 1..17: note = Synthetic: Stx2 I OL2
  source 1..17: mol_type = other DNA
tgtaatattttcttaca

SEQ ID NO: 24 - DNA, 17 bp, synthetic construct
  misc_feature 1..17: note = Synthetic: VT2-SA OL3
  source 1..17: mol_type = other DNA
tacaacgctttgtattt

SEQ ID NO: 25 - DNA, 17 bp, synthetic construct
  misc_feature 1..17: note = Synthetic: Stx2 I OL3
  source 1..17: mol_type = other DNA
gcttcttgaataacagc

SEQ ID NO: 26 - DNA, 12 bp, synthetic construct
  misc_feature 1..12: note = Synthetic
  source 1..12: mol_type = other DNA
ctagctagctag